(19) United States
(12) Patent Application Publication
(10) Pub. No.: US 2022/0142811 A1
(43) Pub. Date: May 12, 2022

(54) DEVICE AND METHOD FOR HELPING PREVENT SNORING AND SLEEP APNEA

(71) Applicant: Joseph Schames, Los Angeles, CA (US)

(72) Inventor: Joseph Schames, Los Angeles, CA (US)

(21) Appl. No.: 17/092,638

(22) Filed: Nov. 9, 2020

(51) Int. Cl. A61F 5/56

(52) U.S. Cl. CPC A61F 5/566

(57) ABSTRACT

A mouth appliance that includes an upper tray having an outer wall, an inner wall and an occlusal wall that cooperate to define an upper teeth space, a lower tray having an outer wall, an inner wall and an occlusal wall that cooperate to define a lower teeth space, a tongue retaining member, and an extension member extending between the inner wall of the upper tray and the tongue retaining member.

### FIELD OF THE INVENTION

The present invention relates generally to a device for helping prevent snoring and sleep apnea.

### BACKGROUND OF THE INVENTION

Snoring and obstructions of the airway during sleep are prevalent problems in the general population. When severe, obstructions of the airway during sleep can cause momentary cessation of breathing. Cessation of breathing lasting at least for 10 seconds is referred to as apnea. Hypopnea is an episode of obstruction of the airway where there is a considerable reduction in the amount of air, but not necessarily a total cessation of breathing.

Snoring may be a loud and vibratory noise produced by turbulent airflow caused by restriction of air passages through partially obstructed airways. The muscle tone of the body ordinarily relaxes during sleep. At the level of the throat, the human airway is composed of collapsible walls of soft tissue which can obstruct breathing during sleep. Snoring can result from the tongue falling backwards onto the soft palate and the pharynx. This is due to relaxation of the muscles that control the throat, back of the tongue, lower jaw, and neck.

Snoring and obstructions of the airway during sleep are associated with significant morbidity, and a higher risk of developing cardiovascular diseases, high blood pressure, and diabetes. Excessive daytime sleepiness resulting from snoring or apnea increases the risk of being involved in traffic accidents as well. Snoring can also interfere with marital harmony.

Some treatments involve lifestyle changes, such as avoiding alcohol or muscle relaxants, losing weight, and/or quitting smoking. Many people benefit from sleeping at a 30 degree elevation of the upper body or higher, as if in a recliner. Doing so, helps prevent the gravitational collapse of the airway. Lateral positions (sleeping on a side), as opposed to supine positions (sleeping on the back), are also recommended as a treatment for snoring and sleep apnea, largely because the gravitational component of the tongue falling back to obstruct the airway is less in the lateral position. There are also surgical procedures to remove and tighten tissue and widen the airway, but these tend to be very intrusive and can have serious risks and complications.

Continuous positive airway pressure (CPAP) is a standard treatment for obstructions of the airway that occur during sleep. Unfortunately, up to 83% of patients that are prescribed CPAP are non-adherent and use their CPAP less than four hours per night.

In spite of the different prior inventions for the control of snoring and sleep apnea, there still remains a need for additional improved methods and devices for reducing the burden of managing snoring and sleep apnea through minimally invasive approaches that provide long term results, that encourage patient compliance, and that minimize patient discomfort.

The floor of the mouth consists of muscles involved in keeping the airway open. The Genioglossus muscle is connected from behind the chin, and becomes the major muscle of the base of the tongue. The Geniohyoid muscle is connected from behind the chin and inserts into the hyoid bone which is a small bone under the mandible and anterior to the trachea airway. The Mylohyoid muscle runs from the mandible to the hyoid bone, forming the floor of the oral cavity of the mouth.

### SUMMARY OF THE PREFERRED EMBODIMENTS

In accordance with a first aspect of the present invention there is provided a mouth appliance that includes an upper tray having an outer wall, an inner wall and an occlusal wall that cooperate to define an upper teeth space, a lower tray having an outer wall, an inner wall and an occlusal wall that cooperate to define a lower teeth space, a tongue retaining member, and an extension member extending between the inner wall of the upper tray and the tongue retaining member.

In a preferred embodiment, the tongue retaining member includes an upper wall, a lower wall and a front wall that cooperate to define a tongue retaining member interior. Preferably, the front wall is curved and defines a tip, the extension member defines first and second ends, and the first end of the extension member is attached to the inner wall of the upper tray and wherein the second end is connected to the tip of the front wall. In another embodiment it can be a flat front wall or surface instead of including a tip. In a preferred embodiment, the inner wall of the upper tray includes an upper surface, and the extension member extends from the upper surface. Preferably, the inner wall of the upper tray includes an inner surface, the extension member includes an upper surface, and the inner surface of the inner wall of the upper tray and the upper surface of the extension member form a continuous surface. In a preferred embodiment, the extension member is curved. However, in another embodiment, the extension member can be straight and can extend from a location on the outer surface of the inner wall.

In a preferred embodiment, the lower wall of the tongue retaining member includes a frenum recess defined therein. Preferably, the tongue retaining member includes a rear surface, and the frenum recess is defined in the lower wall and the rear surface. In a preferred embodiment, at least a portion of the tongue retaining member is positioned higher than the upper tray.

In accordance with another aspect of the present invention there is provided a method of helping prevent snoring that includes the steps of obtaining a mouth appliance that includes an upper tray having an outer wall, an inner wall and an occlusal wall that cooperate to define an upper teeth space, a lower tray having an outer wall, an inner wall and an occlusal wall that cooperate to define a lower teeth space, a tongue retaining member, wherein the tongue retaining member includes an upper wall, a lower wall and a front wall that cooperate to define a tongue retaining member interior, and an extension member extending between the inner wall of the upper tray and the tongue retaining member, and positioning the mouth appliance in an oral cavity that includes upper teeth, lower teeth, a palate and a tongue, such that the upper teeth are received in the upper teeth space and the lower teeth are positioned in the lower teeth space. The method further includes inserting the tongue into the tongue retaining member interior, such that an upper surface of the tongue that is exterior to the tongue retaining member interior is positioned against the palate.

The present invention includes a method of a user wearing an oral appliance where the mandible is advanced forward keeping the airway open by pulling the base of the tongue and the hyoid bone forward.

The Palatoglossus muscle runs from the aponeurosis of the soft palate and inserts into the side of the tongue (see FIG. 2). When the user wears the oral appliance which advances the mandible forward, the Palatoglossus muscles bilaterally are stretched placing tension on the soft palate preventing obstructions of the airway to occur at the upper pharyngeal area of the soft palate. This also prevents the vibration of the soft palate tissues involved in snoring.

The present invention also includes a method whereby the dorsum of the tongue is retained forward, pulling the base of the tongue forward, and preventing the gravitational drifting back of the base of the tongue which would impinge and obstruct the airway. This occurs especially when a person lies supine (on his or her back). The dorsum of the user's tongue also being retained upward against the hard palate stops airflow through the mouth and promotes nasal breathing. Mouth breathing has been documented in the scientific literature to cause obstructions of the airway. Mouth opening and oral breathing during sleep are associated with narrowing of the pharyngeal lumen and decreases in retroglossal diameter, which increase upper airway collapsibility and may lead to airway obstruction.

The present invention is an oral appliance for the prevention of snoring and sleep apnea. This oral appliance opens the airway by bringing the mandible forward and by also retaining the dorsum of the tongue forward and upward against the upper hard palate. In a preferred embodiment, the appliance is a one-piece device molded from a flexible polymer, which has an upper maxillary tray and a lower mandibular tray, as well as a tongue retaining portion attached to the upper maxillary tray. In another embodiment, the appliance can be multiple or a plurality of pieces and can be made of other materials.

In a preferred embodiment, in use, the oral appliance advances the mandible and hyoid bone forward and thereby opens up the posterior pharyngeal airway; and at the same time the oral appliance retains the user's dorsum of the tongue forward and upward against the user's hard palate which elevates the base of the tongue musculature further opening the airway; thus eliminating snoring and obstructions of the airway during sleep.

In a preferred embodiment, the device is an oral appliance that alters the position of the user's mandible by advancing the position of the mandible forward relative to the position of the maxilla, thereby advancing the hyoid bone in a forward direction which results in the opening of the airway. The advancement of the mandible in a forward direction also stretches the palatoglossus muscles whose origin is the soft palate in the back of the mouth. By tensing the soft palate it does not allow an obstruction of the airway to occur at the upper pharyngeal airway. In a preferred embodiment, the oral appliance also retains the dorsum of the tongue forward and upward against the user's upper hard palate raising the base of the tongue forward. Preferably, the device does not allow the tongue to drift back, thereby preventing the posterior portion of the base of the tongue from closing the airway. The dorsum of the tongue being placed up against the hard palate also stops mouth breathing and promotes nasal breathing. Mouth breathing has been documented in the scientific literature to cause obstructions of the airway.

In a preferred embodiment, the present invention embodies a mouthpiece device that includes an upper maxillary tray and a lower mandibular tray. The upper tray includes an inner wall and an outer wall, the lower tray includes an inner wall and an outer wall, and the device (the upper and lower trays) are hinged together bilaterally. In a preferred embodiment, the device further includes a tongue retaining portion on the inner wall of the upper tray.

In a preferred embodiment, the device serves to reduce the effects of snoring or sleep apnea by both bringing the mandible and hyoid bone forward and at the same time the device retains the user's dorsum of the tongue anteriorly and upward against the hard palate. In a preferred embodiment, the device serves to prevent the user from breathing through the mouth by both allowing the upper and lower trays of the device to close completely together, and also by placing and retaining the dorsum of the user's tongue upward against the hard palate.

In a preferred embodiment, the upper tray and the lower tray have holes (preferably circular, but can be non-circular) defined therethrough to allow for the user's saliva to escape. Preferably, the upper tray and the lower tray allow the user to be able to move his or her mandible from side to side. In a preferred embodiment, the inner wall of the lower tray includes a longer portion, such that in use the longer portion extends into a user's lingual vestibule to provide advancement of the user's mandible and by not placing pressure on the user's lower mandibular incisor teeth. Preferably, the upper tray and the lower tray extend at least to a user's upper and lower molar teeth when the device is fully inserted. Preferably, the device of the present invention reduces the effects of bruxism.

### BRIEF DESCRIPTION OF THE DRAWINGS

The invention may be more readily understood by referring to the accompanying drawings in which:

FIG. 1 is a perspective view of a mouth appliance in accordance with a preferred embodiment of the present invention;

FIG. 2 is another perspective view of the mouth appliance of FIG. 1;

FIG. 3 is another perspective view of the mouth appliance of FIG. 1;

FIG. 4 shows a portion of the mouth appliance in cross-section and positioned in a user's mouth; and

FIG. 4A shows a similar cross-section of the mouth appliance as FIG. 4, but with the extension member attached to the tongue retaining portion in a different location.

Like numerals refer to like parts throughout the several views of the drawings.

### DETAILED DESCRIPTION OF THE PREFERRED EMBODIMENTS

The following description and drawings are illustrative and are not to be construed as limiting. Numerous specific details are described to provide a thorough understanding of the disclosure. However, in certain instances, well-known or conventional details are not described in order to avoid obscuring the description. References to one or another embodiment in the present disclosure can be, but not necessarily are, references to the same embodiment; and, such references mean at least one of the embodiments.

Reference in this specification to “one embodiment” or “an embodiment” means that a particular feature, structure, or characteristic described in connection with the embodiment is included in at least one embodiment of the disclosure. Appearances of the phrase “in one embodiment” in various places in the specification do not necessarily refer to the same embodiment, nor are separate or alternative embodiments mutually exclusive of other embodiments. Moreover, various features are described which may be exhibited by some embodiments and not by others. Similarly, various requirements are described which may be requirements for some embodiments but not other embodiments.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. For convenience, certain terms may be highlighted, for example using italics and/or quotation marks: The use of highlighting has no influence on the scope and meaning of a term; the scope and meaning of a term is the same, in the same context, whether or not it is highlighted. It will be appreciated that the same thing can be said in more than one way.

Consequently, alternative language and synonyms may be used for any one or more of the terms discussed herein. Nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various embodiments given in this specification.

Without intent to further limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

It will be appreciated that terms such as “front,” “back,” “top,” “bottom,” “side,” “short,” “long,” “up,” “down,” and “below” used herein are merely for ease of description and refer to the orientation of the components as shown in the figures. It should be understood that any orientation of the components described herein is within the scope of the present invention.

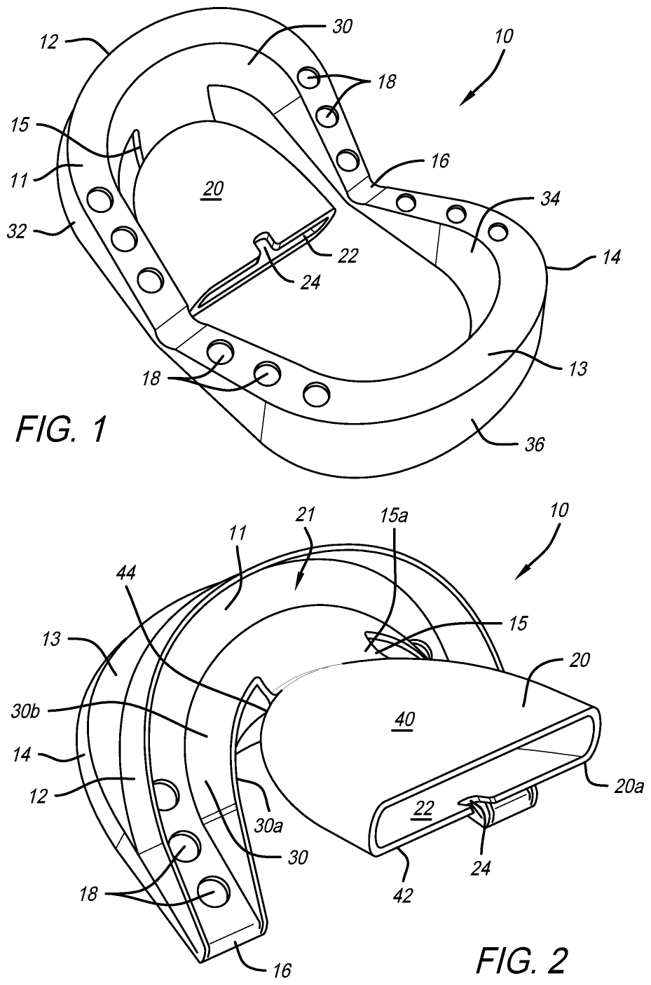

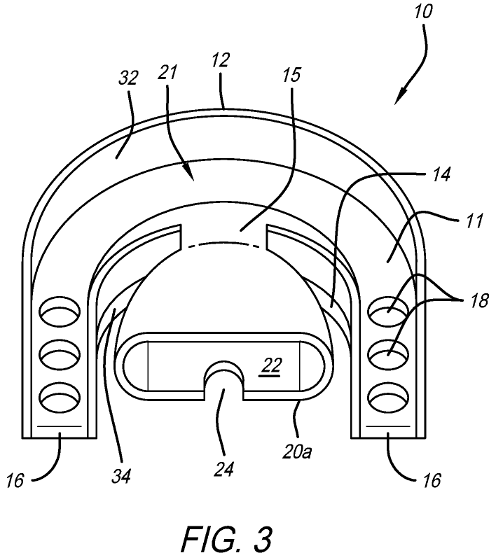

Described herein is a mouth appliance 10 and system for helping prevent snoring and sleep apnea. As shown in FIGS. 1-3, the mouth appliance 10 generally includes an upper tray 12, a lower tray 14, an extension member 15 and a tongue retaining member 20. mber.

FIG. 1 is a perspective view of the preferred embodiment of the invention in an open position, not in the user's mouth. As can be seen, the mouth appliance 10 folds at hinge 16 on the right, and at the hinge 16 on the left. The upper maxillary tray 12 and lower mandibular tray 14 are both u-shaped, joined together by the hinges 16 bilaterally. The length of the upper tray 12 and the lower tray 14 are preferably formed so that the trays 12 and 14 of the device extend back at least to the user's upper and lower molar teeth bilaterally when fully inserted. This allows for an ample amount of space for the teeth to be retained in the upper 12 and lower 14 trays, but does not extend too far into the mouth so as to create discomfort or to promote gagging. The bilateral hinges 16 permit the device to flex and bend as a hinge. The bilateral hinges 16 also allow for lateral movement of the bottom surface of the upper occlusal wall 11 of the upper tray 12 to slide on the top surface of the lower occlusal wall 13 of the lower tray 14 from side to side. The saliva holes 18 that extend through the upper and lower occlusal walls 11 and 13 allow for saliva to escape when the device 10 is in the mouth. In a preferred embodiment, the hinge can be omitted and the upper and lower occlusal walls can be connected to one another (or be a single occlusal wall that includes upper and lower surfaces). Both of the upper and lower walls including an occlusal wall can be a single or combined occlusal wall in an embodiment where the hinges are omitted. In this embodiment, the upper and lower trays can be a U-shape, as shown in the drawings, or can be shaped to only receive the front or anterior teeth. In other words, the upper and lower trays can be a single piece and are not hingedly connected.

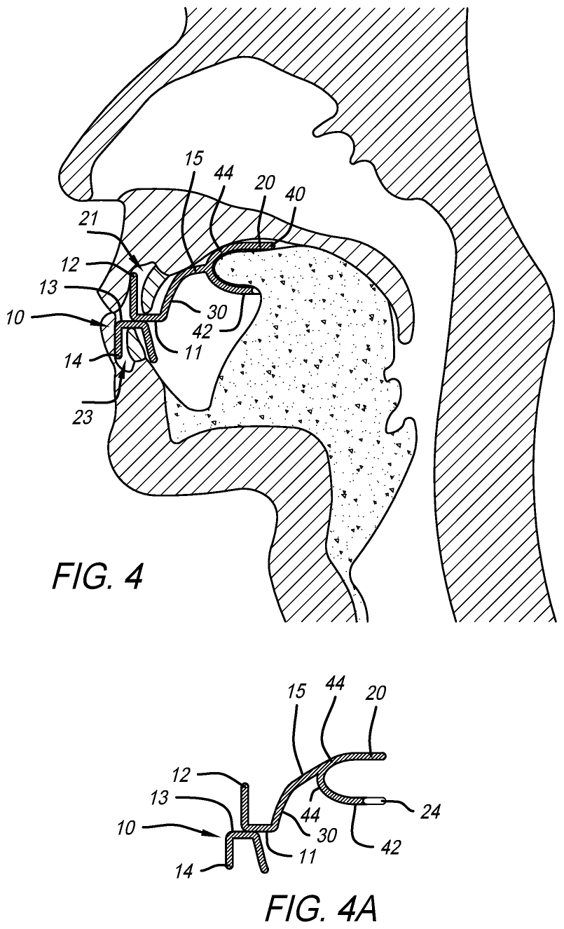

As shown in FIGS. 1-4, in a preferred embodiment, the upper tray 12 includes an outer wall 32, an inner wall 30 and upper occlusal wall 11 that cooperate to define an upper teeth space 21. The lower tray 14 includes an outer wall 36, an inner wall 34 and lower occlusal wall 13 that cooperate to define a lower teeth space 23. Preferably, the extension member 15 extends between the inner wall 30 of the upper tray and the tongue retaining member 20, which extends upward toward the hard palate allowing the tongue to be retained in an anterior and upward position against the hard palate (see FIG. 4). In another embodiment, the tongue retaining member 20 can be directly connected to the top of the inner wall 30 of the upper tray 12. FIG. 1 is a perspective view of the device, showing the upper tray 12 and the lower tray 14 in an open position, with the saliva holes 18 in the upper occlusal wall 11 of the upper tray 12 and on lower occlusal wall 13 of the lower tray 14.

As shown in FIG. 2, the tongue retaining member 20 includes an upper wall 40, a lower wall 42 and a front wall 44 that cooperate to define a tongue retaining member interior 22. The tongue is placed in the tongue retaining member interior 22 opening of the tongue retaining portion 20 of the device 10. In a preferred embodiment, the front wall 44 is curved and defines a tip. Preferably, the extension member 15 defines first and second ends and the first end of the extension member 15 is attached to the inner wall 30 of the upper tray 12 and the second end is connected to the tip of the front wall 44. In a preferred embodiment, as is best shown in FIG. 2, the inner wall 30 of the upper tray 12 includes an upper surface 30a, and the extension member 15 extends from the upper surface 30a. The inner wall 30 of the upper tray 12 includes an inner surface 30b, the extension member includes an upper surface 15a, and the inner surface 30b of the inner wall 30 of the upper tray 12 and the upper surface 15a of the extension member 15 form a continuous surface. In another embodiment, the extension member can extend from the outer surface of the inner wall. As shown in FIG. 4A, in another embodiment, the extension member 15 can extend to a location at the top of the front wall 44 or blend into the upper wall 40 of the tongue retaining member 20.

As shown in FIG. 4, in a preferred embodiment, the extension member 15 is curved and such that at least a portion of the tongue retaining member 20 is positioned horizontally above the upper tray 12. As will be appreciated by a person of ordinary skill in the art, the palate curves upwardly. The curved and projecting upwardly extension member 15 allows the tongue retaining member 20 to be positioned near or against the palate.

Anatomically, the lower portion of the tongue has an attached tissue connecting the tongue to the bottom of the floor of the mouth called a frenum. In a preferred embodiment, the lower wall 42 of the tongue retaining member includes a frenum recess frenum cleft or frenum recess 24 defined therein. The tongue retaining member 20 includes a rear surface 20a. Preferably, the frenum recess 24 is defined in the lower wall 42 and the rear surface 20a. The frenum recess allows the frenum to extend therethrough so that it is not irritated when the tongue is held in the tongue retaining member 20. The frenum recess 24 can be U or V-shaped.

In a preferred embodiment, the inner wall 34 of the lower mandibular tray 14 includes a longer lingual portion that serves to extend into the user's lingual vestibule below the user's lower incisor teeth to advance the mandible forward by pressure placed on the user's lingual vestibule portion of the mandible instead of pressure placed directly on the user's teeth.

In a preferred embodiment, the lower tray 14 is extends forward of the upper tray 12. This is to bring the user's mandible forward to open the airway. The tongue retaining portion 20 is preferably extends toward the user's palate as it is connected to the extension portion 15 or the inner wall 30 of the upper tray 12. The hinge 16 is closed position when the upper maxillary tray 12 is closed onto the lower mandibular tray 14, as shown in FIG. 2.

Overall mouth appliance 10 readily bends in half at bilateral hinges 16. In use, the mouth appliance 10 is inserted into the user's mouth or oral cavity, with the user's maxillary teeth pressing into upper maxillary tray 12 and the user's mandibular teeth pressing into lower mandibular tray 14. The user's maxillary teeth are cradled by the inner wall of upper maxillary tray 30 and the outer wall of upper maxillary tray 32. The user's mandibular teeth are cradled by the inner wall 34 of lower mandibular tray 14, and the outer wall 36 of the lower mandibular tray 14. The lower tray 14 protruding further than the upper tray 12 maintains the mandible in a forward position. In addition, the contact of the longer lingual inner wall of tray 14 to the lingual surface of the lower lingual vestibule maintains the mandible in a forward position relative to the maxilla, whereby the airway passage in the pharyngeal airway is kept open thus eliminating snoring and/or obstructions of the airway. At the same time, the user's tongue is held by a vacuum surface tension of the tongue in the tongue retaining portion 20 of the mouth appliance 10. In this way the tongue is held anteriorly and upward against the palate, as shown in FIG. 4. This blocks any mouth breathing and also retains the tongue from falling back and closing the pharyngeal airway, thus also eliminating snoring and/or obstructions of the airway.

The position of the two closed trays 12 and 14 relative to each other, holds the mandible forward and still allows side to side motion between the upper and lower trays 12 and 14, which makes it more comfortable for the user. It also allows the user to have his or her mouth closed to prevent any mouth breathing which can cause obstructions of the airway to occur.

In the preferred embodiment, the mouth appliance is molded from an appropriate suitable flexible material, whether in the form of synthetic polymers, silicone, or even natural rubber.

While the overall mouth appliance serves primarily for the purpose of mandibular advancement, with the associated sleep-related benefits, the device also serves to reduce problems associated with bruxism, or the grinding of the teeth. The mechanism for this is readily apparent, as the upper and lower trays prevent the maxillary and mandibular teeth from making direct contact.

The above described elements combine to create a mandibular advancement and a tongue retaining device that does not require custom fitting and is easily used off-the-shelf. Although the above specification and related drawings describe a specific embodiment of the invention, this should not be interpreted as limiting the scope of the invention to these particular elements. The interpretation of the scope of the invention should be determined by the claims and their legal equivalents.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description of the Preferred Embodiments using the singular or plural number may also include the plural or singular number respectively. The word “or” in reference to a list of two or more items, covers all of the following interpretations of the word: any of the items in the list, all of the items in the list, and any combination of the items in the list.

Embodiments are envisioned where any of the aspects, features, component or steps herein may be omitted and/or are option. Furthermore, where appropriate any of these optional aspects, features, component or steps discussed herein in relation to one aspect of the invention may be applied to another aspect of the invention.

The above-detailed description of embodiments of the disclosure is not intended to be exhaustive or to limit the teachings to the precise form disclosed above. While specific embodiments of and examples for the disclosure are described above for illustrative purposes, various equivalent modifications are possible within the scope of the disclosure, as those skilled in the relevant art will recognize. For example, while processes or blocks are presented in a given order, alternative embodiments may perform routines having steps, or employ systems having blocks, in a different order, and some processes or blocks may be deleted, moved, added, subdivided, combined, and/or modified to provide alternative or subcombinations. Each of these processes or blocks may be implemented in a variety of different ways. Also, while processes or blocks are at times shown as being performed in series, these processes or blocks may instead be performed in parallel, or may be performed, at different times. Further any specific numbers noted herein are only examples: alternative implementations may employ differing values or ranges.

The above-detailed description of embodiments of the disclosure is not intended to be exhaustive or to limit the teachings to the precise form disclosed above. While specific embodiments of and examples for the disclosure are described above for illustrative purposes, various equivalent modifications are possible within the scope of the disclosure, as those skilled in the relevant art will recognize. Further, any specific numbers noted herein are only examples: alternative implementations may employ differing values, measurements or ranges. It will be appreciated that any dimensions given herein are only exemplary and that none of the dimensions or descriptions are limiting on the present invention.

The teachings of the disclosure provided herein can be applied to other systems, not necessarily the system described above. The elements and acts of the various embodiments described above can be combined to provide further embodiments.

Any patents and applications and other references noted above, including any that may be listed in accompanying filing papers, are incorporated herein by reference in their entirety. Aspects of the disclosure can be modified, if necessary, to employ the systems, functions, and concepts of the various references described above to provide yet further embodiments of the disclosure.

These and other changes can be made to the disclosure in light of the above Detailed Description of the Preferred Embodiments. While the above description describes certain embodiments of the disclosure, and describes the best mode contemplated, no matter how detailed the above appears in text, the teachings can be practiced in many ways. Details of the system may vary considerably in its implementation details, while still being encompassed by the subject matter disclosed herein. As noted above, particular terminology used when describing certain features or aspects of the disclosure should not be taken to imply that the terminology is being redefined herein to be restricted to any specific characteristics, features or aspects of the disclosure with which that terminology is associated. In general, the terms used in the following claims should not be construed to limit the disclosures to the specific embodiments disclosed in the specification unless the above Detailed Description of the Preferred Embodiments section explicitly defines such terms. Accordingly, the actual scope of the disclosure encompasses not only the disclosed embodiments, but also all equivalent ways of practicing or implementing the disclosure under the claims.

While certain aspects of the disclosure are presented below in certain claim forms, the inventors contemplate the various aspects of the disclosure in any number of claim forms. For example, while only one aspect of the disclosure is recited as a means-plus-function claim under 35 U.S.C. § 112, ¶6, other aspects may likewise be embodied as a means-plus-function claim, or in other forms, such as being embodied in a computer-readable medium. (Any claims intended to be treated under 35 U.S.C. § 112, ¶6 will begin with the words “means for”). Accordingly, the applicant reserves the right to add additional claims after filing the application to pursue such additional claim forms for other aspects of the disclosure.

Accordingly, although exemplary embodiments of the invention have been shown and described, it is to be understood that all the terms used herein are descriptive rather than limiting, and that many changes, modifications, and substitutions may be made by one having ordinary skill in the art without departing from the spirit and scope of the invention.

What is claimed is:

1. A mouth appliance comprising:
an upper tray that includes an outer wall, an inner wall and an occlusal wall that cooperate to define an upper teeth space, wherein the inner wall includes an upper surface,
a lower tray that includes an outer wall, an inner wall and an occlusal wall that cooperate to define a lower teeth space, wherein the upper tray and the lower tray are hingedly connected,
a tongue retaining member, wherein the tongue retaining member includes an upper wall, a lower wall and a front wall that cooperate to define a tongue retaining member interior, wherein the front wall is curved and defines a tip, wherein the tongue retaining member includes a rear surface, and wherein a frenum recess is defined in the lower wall and the rear surface, and
a curved extension member extending between the inner wall of the upper tray and the tongue retaining member, wherein the curved extension member defines first and second ends, wherein the first end of the curved extension member extends from the upper surface the inner wall of the upper tray and wherein the second end is connected to the front wall at a location above the tip.

2. The mouth appliance of claim 1 wherein the lower tray and upper tray are configured such that when the oral appliance is worn a user's mandible is advanced forwardly.

3. The mouth appliance of claim 2 wherein the lower tray is movable between a closed position and an open position, and wherein when the lower tray is in the closed position, the outer wall of the lower tray is positioned forwardly of the outer wall of the upper tray.

4. (canceled)

5. The mouth appliance of claim 3 wherein the inner wall of the upper tray includes an inner surface and is inclined rearwardly with respect to the occlusal wall, wherein the curved extension member includes an upper surface, and wherein the inner surface of the inner wall of the upper tray, the upper surface of the curved extension member and an upper surface of the upper wall of the tongue retaining member form a continuous surface, such that the upper wall of the curved extension member blends in with the upper surface of the upper wall of the tongue retaining member to approximate a curvature of the palate.

6-7. (canceled)

8. The mouth appliance of claim 5 wherein the tongue retaining member includes a rear surface, and wherein a frenum recess is defined in the lower wall and the rear surface of the tongue retaining member.

9. The mouth appliance of claim 8 wherein the upper wall of the tongue retaining member is positioned higher than an upper surface of the outer wall of the upper tray.

10. The mouth appliance of claim 9 wherein the occlusal walls of the upper and lower trays include saliva holes defined therethrough.

11-12. (canceled)

13. A method of helping prevent snoring comprising the steps of:
obtaining a mouth appliance that includes an upper tray having an outer wall, an inner wall and an occlusal wall that cooperate to define an upper teeth space, a lower tray having an outer wall, an inner wall and an occlusal wall that cooperate to define a lower teeth space, wherein the upper tray and the lower tray are hingedly connected, wherein the lower tray is movable between a closed position and an open position, a tongue retaining member, wherein the tongue retaining member includes an upper wall, a lower wall and a front wall that cooperate to define a tongue retaining member interior, and an extension member extending between the inner wall of the upper tray and the tongue retaining member,
positioning the mouth appliance in an oral cavity that includes upper teeth, lower teeth that are part of a mandible, a palate and a tongue, such that the upper teeth are received in the upper teeth space and the lower teeth are positioned in the lower teeth space,
inserting the tongue into the tongue retaining member interior, whereby an upper surface of a portion of the tongue that is positioned exterior to the tongue retaining member interior is positioned against the palate, and
moving the mandible from a closed position to an open position to move the lower tray to the open position, wherein the lower teeth are still received in the lower teeth space.

14. The method of claim 13 wherein the front wall is curved and defines a tip, wherein the extension member defines first and second ends, wherein the first end of the extension member is attached to the inner wall of the upper tray and wherein the second end is connected to the tip of the front wall.

15. The method of claim 14 wherein the inner wall of the upper tray includes an upper surface, and wherein the extension member extends from the upper surface.

16. The method of claim 15 wherein the inner wall of the upper tray includes an inner surface, wherein the extension member includes an upper surface, and wherein the inner wall of the upper tray and the upper surface of the extension member form a continuous surface.

17. The method of claim 16 wherein the extension member is curved to approximate a curvature of the palate.

18. The method of claim 13 wherein the lower wall of the tongue retaining member includes a frenum recess defined therein and a frenum of the tongue is positioned in the frenum recess.

19. (canceled)
